(19) United States
(12) Patent Application Publication
(10) Pub. No.: US 2025/0302481 A1
(43) Pub. Date: Oct. 2, 2025

(54) ENDOBRONCHIAL BLOCKER SYSTEM AND APPARATUS

(71) Applicant: Yale University, New Haven, CT (US)

(72) Inventor: Peter Kahn, New Haven, CT (US)

(21) Appl. No.: 18/616,372

(22) Filed: Mar. 26, 2024

(51) Int. Cl. A61B 17/12; A61B 17/00; A61B 90/00; A61M 25/00; A61M 25/01; A61M 25/10

(52) U.S. Cl. CPC A61B 17/12136; A61B 17/12045; A61B 17/12104; A61B 2017/00106; A61B 2017/12004; A61B 2017/1205; A61B 2090/064; A61M 2025/0036; A61M 2025/0039; A61M 2025/004; A61M 25/0102; A61M 25/1011; A61M 2025/1052

(57) ABSTRACT

A catheter includes an elongated member having a distal end and a proximal end with a plurality of lumens therein. A first lumen extends to a first opening at the distal end. A first balloon is disposed about the elongated member adjacent the distal end, and a second lumen extends to a second opening at an interior of the first balloon for inflating the first balloon. A second balloon is disposed about the elongated member at a position proximal from the first balloon forming a first gap therebetween, and a third lumen extends to a third opening at an interior of the second balloon for inflating the second balloon. A fourth lumen extends to a fourth opening in the first gap for positioning a first sensor. A fifth lumen extends to a fifth opening at a position proximal from the second balloon for positioning a second sensor.

### FIELD

The present technology relates generally to the field of medical devices, and more particularly, to a multi-balloon catheter for blocking a passageway and/or removing an obstruction from the passageway.

### BACKGROUND

Hemoptysis is the discharge of blood or blood-stained mucus through the mouth coming from the bronchi, larynx, trachea, or lungs. Hemoptysis that occurs in patients who are critically ill or undergoing medical procedures, such as bronchoscopy, can seriously impede the medical procedure and can be life-threatening for the patient. One treatment for hemoptysis includes inserting a catheter with an inflatable balloon into the bleeding airway, inflating the balloon to block the airway, and waiting for a clot to form, thus stopping the bleeding.

However, traditional balloon catheters are unable to obtain and provide information to the patient's medical provider about what is happening in the airway distal to the catheter. Furthermore, traditional balloon catheters also lack a failsafe for when the singular deployed balloon does not work, leaks, or is overwhelmed by pressure in the airway. In such a scenario of balloon failure, the balloon must be left in place, deflated, or removed before further treatment methods can be implemented. During this time, additional blood or undesirable fluids can flow past the area intended to be obstructed. Additionally, traditional balloon catheters do not provide a means for the delivery of therapies to the airway distal to the balloon and do not provide a means to retrieve and remove undesirable objects from the airway.

What is needed, therefore, are improved balloon catheters and hemoptysis treatment methods that address at least some of the problems described above.

### SUMMARY

According to an embodiment of the present technology, a catheter is provided. The catheter includes an elongated member with a plurality of lumens therein. The elongated member has a distal end and a proximal end defining a length of the elongated member. A first of the plurality of lumens extends to a first opening at the distal end of the elongated member. A first inflatable balloon is disposed about the elongated member adjacent the distal end, and a second of the plurality of lumens extends to a second opening at an interior of the first inflatable balloon for inflating the first inflatable balloon. A second inflatable balloon is disposed about the elongated member at a position proximal from the first inflatable balloon forming a first gap between the first inflatable balloon and the second inflatable balloon, and a third of the plurality of lumens extends to a third opening at an interior of the second inflatable balloon for inflating the second inflatable balloon. A fourth of the plurality of lumens extends to a fourth opening in the first gap for positioning at least one first sensor. A fifth of the plurality of lumens extends to a fifth opening at a position proximal from the second inflatable balloon for positioning at least one second sensor.

In some embodiments, each of the at least one first sensor and the at least one second sensor are configured to measure fluid characteristics in a passageway when the catheter is positioned in the passageway.

In some embodiments, a third inflatable balloon is disposed about the elongated member at a position proximal from the second inflatable balloon forming a second gap between the second inflatable balloon and the third inflatable balloon. A sixth of the plurality of lumens extends to a sixth opening at an interior of the third inflatable balloon for inflating the third inflatable balloon. A seventh of the plurality of lumens extends to a seventh opening at a position proximal from the third inflatable balloon for positioning at least one third sensor. The fifth opening for positioning the at least one second sensor via the fifth lumen is positioned in the second gap.

In some embodiments, each of the at least one first sensor and the at least one second sensor include pressure transducers, ultrasound transducers, sonic tips, flow sensors, or combinations thereof.

In some embodiments, the first lumen is configured for delivering at least one medication or medical tool to a position distal from the distal end of the elongated member.

In some embodiments, an eighth of the plurality of lumens extends to an eighth opening at the distal end of the elongated member for positioning at least one fourth sensor.

In some embodiments, a ninth of the plurality of lumens extends at least partially through the elongated member for receiving a stiffening member therein.

In some embodiments, at least two of the plurality of lumens are parallel to each other in a side-by-side arrangement.

In some embodiments, at least two of the plurality of lumens are in a coaxial arrangement.

According to another embodiment of the present technology, a system for blocking a passageway is provided. The system includes a catheter that is configured for insertion into the passageway, a first inflation device, and a second inflation device. The catheter includes an elongated member with a plurality of lumens therein. The elongated member has a distal end and a proximal end defining a length of the elongated member. A first of the plurality of lumens extends to a first opening at the distal end of the elongated member. A first inflatable balloon is disposed about the elongated member adjacent the distal end, and a second of the plurality of lumens extends to a second opening at an interior of the first inflatable balloon for inflating the first inflatable balloon. A second inflatable balloon is disposed about the elongated member at a position proximal from the first inflatable balloon forming a first gap between the first inflatable balloon and the second inflatable balloon, and a third of the plurality of lumens extends to a third opening at an interior of the second inflatable balloon for inflating the second inflatable balloon. A fourth of the plurality of lumens extends to a fourth opening in the first gap for positioning at least one first sensor. A fifth of the plurality of lumens extends to a fifth opening at a position proximal from the second inflatable balloon for positioning at least one second sensor. The first inflation device is in fluid communication with the second lumen and is configured to deliver a first fluid into the second lumen to inflate the first inflatable balloon and block fluid flow in the passageway. The second inflation device is in fluid communication with the third lumen and is configured to deliver a second fluid into the second lumen to inflate the second inflatable balloon and block fluid flow in the passageway.

In some embodiments, each of the at least one first sensor and the at least one second sensor are configured to measure fluid characteristics in the passageway. In some embodiments, a third inflatable balloon is disposed about the elongated member at a position proximal from the second inflatable balloon forming a second gap between the second inflatable balloon and the third inflatable balloon. A sixth of the plurality of lumens extends to a sixth opening at an interior of the third inflatable balloon for inflating the third inflatable balloon. A seventh of the plurality of lumens extends to a seventh opening at a position proximal from the third inflatable balloon for positioning at least one third sensor. The at least one third sensor is configured to measure fluid characteristics in the passageway. The fifth opening for positioning the at least one second sensor via the fifth lumen is positioned in the second gap.

In some embodiments, a third inflation device is in fluid communication with the sixth lumen. The third inflation device is configured to deliver a third fluid into the sixth lumen to inflate the third inflatable balloon and block fluid flow in the passageway.

In some embodiments, each of the at least one first sensor, the at least one second sensor, and the at least one third sensor include pressure transducers, ultrasound transducers, sonic tips, flow sensors, or combinations thereof.

In some embodiments, the first lumen is configured for delivering at least one medication or medical instrument to a position in the passageway distal from the distal end of the elongated member.

According to another embodiment of the present technology, a method for blocking a passageway is provided. The method includes inserting a catheter into a passageway, the catheter includes an elongated member with a plurality of lumens therein, the elongated member having a distal end and a proximal end defining a length of the elongated member, a first of the plurality of lumens extending to a first opening at the distal end of the elongated member; a first inflatable balloon disposed about the elongated member adjacent the distal end, a second of the plurality of lumens extending to a second opening at an interior of the first inflatable balloon for inflating the first inflatable balloon; a second inflatable balloon disposed about the elongated member at a position proximal from the first inflatable balloon forming a first gap between the first inflatable balloon and the second inflatable balloon, a third of the plurality of lumens extending to a third opening at an interior of the second inflatable balloon for inflating the second inflatable balloon; a fourth of the plurality of lumens extending to a fourth opening in the first gap for positioning at least one first sensor; and a fifth of the plurality of lumens extending to a fifth opening at a position proximal from the second inflatable balloon for positioning at least one second sensor. Each of the at least one first sensor and the at least one second sensor are configured to measure fluid characteristics in the passageway. The method includes positioning the catheter in the passageway such that the distal end of the elongated member is adjacent a desired location; and inflating, via a first inflation device in fluid communication with the first inflatable balloon, the first inflatable balloon with a first fluid to block fluid flow in the passageway.

In some embodiments, the method further includes detecting, via the at least one first sensor, fluid characteristics in the passageway adjacent the first inflatable balloon; assessing fluid characteristics data received from the at least one first sensor; and inflating, via a second inflation device in fluid communication with the second inflatable balloon, the second inflatable balloon with a second fluid to block fluid flow in the passageway if the fluid characteristics adjacent the first inflatable balloon reaches a predetermined threshold.

In some embodiments, the catheter further includes a third inflatable balloon that is disposed about the elongated member at a position proximal from the second inflatable balloon forming a second gap between the second inflatable balloon and the third inflatable balloon. A sixth of the plurality of lumens extends to a sixth opening at an interior of the third inflatable balloon for inflating the third inflatable balloon. A seventh of the plurality of lumens extends to a seventh opening at a position proximal from the third inflatable balloon for positioning at least one third sensor. The at least one third sensor is configured to measure fluid characteristics in the passageway. The fifth opening for positioning the at least one second sensor via the fifth lumen is positioned in the second gap.

In some embodiments, the method further includes detecting, via the at least one second sensor, fluid characteristics in the passageway adjacent the second inflatable balloon; assessing fluid characteristics data received from the at least one second sensor; and inflating, via a third inflation device in fluid communication with the third inflatable balloon, the third inflatable balloon with a third fluid to block fluid flow in the passageway if the fluid characteristics adjacent the second inflatable balloon reaches a predetermined threshold.

In some embodiments, the method further includes detecting, via the at least one third sensor, fluid characteristics in the passageway adjacent the third inflatable balloon; and assessing fluid characteristics data received from the at least one third sensor to monitor for the fluid characteristics adjacent the third inflatable balloon reaching a predetermined threshold.

In some embodiments, the catheter is positioned in the passageway such that the first inflatable balloon is positioned distal from an obstruction in the passageway and the second inflatable balloon is positioned proximal from the obstruction. The method further includes inflating, via a second inflation device in fluid communication with the second inflatable balloon, the second inflatable balloon with a second fluid to trap the obstruction in the first gap between the first inflatable balloon and the second inflatable balloon; and removing the catheter from the passageway with the obstruction trapped in the first gap.

Further objects, aspects, features, and embodiments of the present technology will be apparent from the drawing Figures and below description.

### BRIEF DESCRIPTION OF DRAWINGS

Some embodiments of the present technology are illustrated as an example and are not limited by the figures of the accompanying drawings, in which like references may indicate similar elements.

FIG. 1 is a side partial cross-sectional view of a balloon catheter according to some embodiments of the present technology.

FIG. 2 is a side view of the balloon catheter of FIG. 1 with the balloon in an inflated state.

FIG. 3 is a cross-sectional view of a balloon catheter according to some embodiments of the present technology taken along section line A-A′ of FIG. 1.

FIG. 4 is a cross-sectional view of a balloon catheter according to some embodiments of the present technology taken along section line A-A′ of FIG. 1.

FIG. 5 is a flowchart of a method of blocking a passageway according to some embodiments of the present technology.

### DETAILED DESCRIPTION

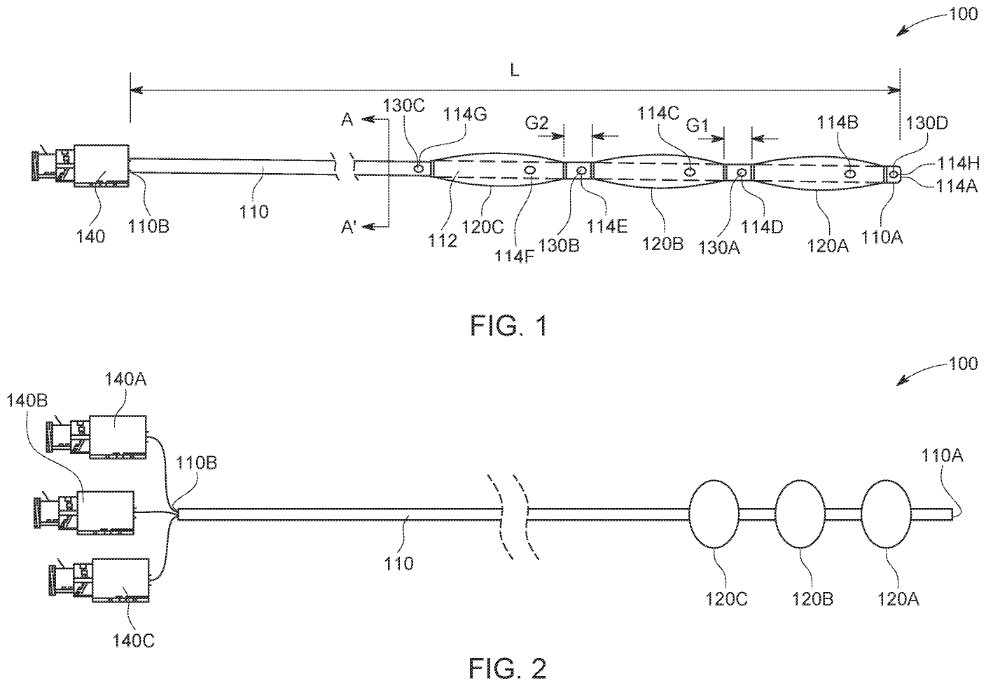

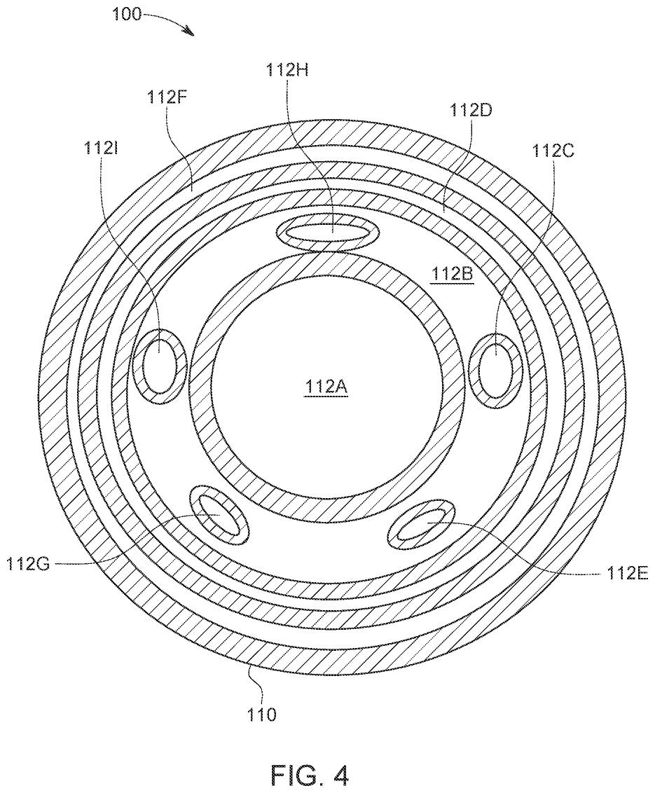

FIGS. 1-3 illustrate a catheter 100 according to some embodiments of the present technology. The catheter 100 includes an elongated member 110 that has a distal end 110A and a proximal end 110B that define a length L of the elongated member 110. The catheter 100 is configured to be inserted into a passageway, such as the tubular passageway within the body of a living organism (e.g., endobronchial passageways, pulmonary passageways, urethra, etc., of a human, dog, cat, horse, cow, etc.). Although in some embodiments the catheter 100 is inserted into a tubular passageway, the present technology is not limited thereto and contemplates embodiments where the catheter 100 is inserted into a passageway having any other cross-sectional shape, such as triangular, rectangular, pentagonal, hexagonal, etc. The elongated member 110 has a plurality of lumens 112 therein. As discussed herein, each lumen of the plurality of lumens is referred to numerically (i.e., first lumen, second lumen, third lumen, etc.). However, this is intended only to differentiate between each of the plurality of lumens and is not intended to limit the present technology to any particular number or ordering of the lumens. In some embodiments, two or more of the plurality of lumens 112 run parallel to each other in a side-by-side arrangement substantially along the length L of the elongated member 110, as shown in FIG. 3. In some embodiments, two or more of the plurality of lumens 112 are in a coaxial arrangement substantially along the length L of the elongated member 110, as shown in FIG. 4.

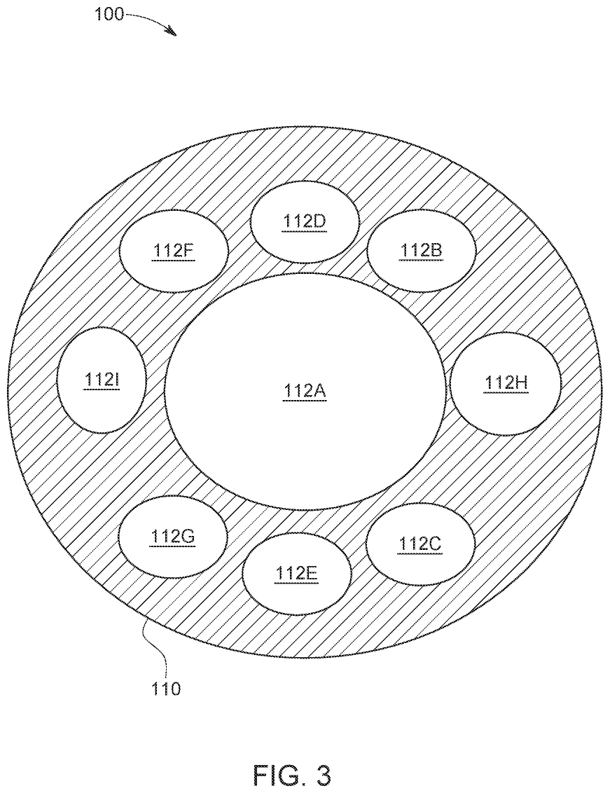

As shown in FIGS. 1-2, the catheter 100 includes a plurality of inflatable balloons 120 disposed about the elongated member 110. The balloons 120 are configured to be inflated to block fluid flow (e.g., blood) within the passageway and/or trap and remove an obstruction from the passageway. As used herein, the term “obstruction” refers to an undesirable object within the passageway that at least partially blocks the passageway. As used herein, the term “undesirable object” refers to a growth, a foreign object, etc., that is not desired to remain within the passageway. FIG. 1 shows the plurality of inflatable balloons 120 in an uninflated state, and FIG. 2 shows the plurality of inflatable balloons 120 in an inflated state. In some embodiments, the inflatable balloons 120 each have a substantially spherical and torus shape, as shown in FIG. 2. However, the present technology is not limited thereto and contemplates embodiments where the inflatable balloons 120 have different shapes, such as toroidal, cylindrical, rectangular, hexagonal, etc. The catheter 100 includes a first inflatable balloon 120A that is disposed about the elongated member 110 at a position at or adjacent to the distal end 110A. A second inflatable balloon 120B is disposed about the elongated member 110 at a position proximal from the first inflatable balloon 120A such that there is a first gap G1 between the first inflatable balloon 120A and the second inflatable balloon 120B. In some embodiments, the catheter 100 includes a third inflatable balloon 120C that is disposed about the elongated member 110 at a positioned proximal from the second inflatable balloon 120B such that there is a second gap G2 between the second inflatable balloon 120B and the third inflatable balloon 120C. Thus, the plurality of inflatable balloons 120 are arranged in series along the length L of the elongated member 110. Although the catheter shown in the drawing figures includes three inflatable balloons, the present technology is not limited thereto and contemplates embodiments where the catheter includes any number of balloons, such as two, four, five, six, etc.

In some embodiments, a first lumen 112A of the plurality of lumens 112 extends within the elongated member 110 to a first opening 114A at the distal end 110A of the elongated member 110. The first lumen 112A is configured to receive one or more medications and/or medical tools therein for delivery and/or use at a position distal from the distal end 110A of the elongated member 110. Thus, the first lumen 112A may also be referred to as the “working” lumen. In some embodiments, the medications delivered via the first lumen 112A are used to treat hemoptysis or other medical conditions (such as the retrieval and removal of kidney stones and other undesirable objects, endoscopic retrograde cholangiopancreatography procedures to diagnose and treat problems in the liver, gallbladder, bile ducts, and pancreas, etc.) and include saline solutions at various temperatures (e.g., chilled, room temperature, heated), tranexamic acid, epinephrine, vasopressin, thrombin, cellulose or other surgical mesh, glue (e.g., cyanoacetate glue, cyanoacrylate glue, etc.), etc., or combinations thereof. In some embodiments, the medical tools used via the first lumen 112A include forceps, brushes, aspiration or biopsy needles, argon plasma coagulation tools, cryo-tools, laser tools, additional balloon tools, suction catheters, miniature bronchoscope or fiberoptic cameras, endobronchial ultrasound (EBUS) or other ultrasound probes, etc., or combinations thereof.

A second lumen 112B of the plurality of lumens 112 extends within the elongated member 110 to a second opening 114B at an interior of the first inflatable balloon 120A. The second lumen 112B is configured to deliver a first fluid (e.g., air, water, etc.) to the interior of the first inflatable balloon 120A for inflating the first inflatable balloon 120A. A third lumen 112C of the plurality of lumens 112 extends within the elongate member 110 to a third opening 114C at an interior of the second inflatable balloon 120B. The third lumen 112C is configured to deliver a second fluid (e.g., air, water, etc.) to the interior of the second inflatable balloon 120B for inflating the second inflatable balloon 120B.

The catheter 100 includes a plurality of sensors 130 positioned throughout the elongated member 110. The sensors 130 are configured to measure fluid characteristics in the passageway, such as fluid pressure, flow, quantity, clot accumulation and density, etc. In some embodiments, the sensors 130 include pressure transducers, sonic tips, ultrasound transducers, flow sensors, etc., or combinations thereof. In some embodiments, at least one first sensor 130A is positioned in a fourth opening 114D in the first gap G1 between the first inflatable balloon 120A and the second inflatable balloon 120B via a fourth lumen 112D within the elongated member 110. In some embodiments, at least one second sensor 130B is positioned proximal from the second inflatable balloon 120B in a fifth opening 114E, such as in the second gap G2 between the second inflatable balloon 120B and the third inflatable balloon 120C, via a fifth lumen 112E within the elongated member 110.

In some embodiments, a sixth lumen 112F of the plurality of lumens 112 extends within the elongated member 110 to a sixth opening 114F at an interior of the third inflatable balloon 120C. The sixth lumen 112F is configured to deliver a third fluid (e.g., air, water, etc.) to the interior of the third inflatable balloon 120C for inflating the third inflatable balloon 120C. At least one third sensor 130C is positioned proximal from the third inflatable balloon 120C in a seventh opening 114G via a seventh lumen 112G within the elongated member 110. In some embodiments, the seventh opening 114G is positioned proximal from and adjacent to the third inflatable balloon 120C such that the at least one third sensor 130C is positioned in a third gap between the third inflatable balloon and a fourth inflatable balloon in embodiments having more than three inflatable balloons (not shown).

At least one fourth sensor 130D is positioned distal from the first inflatable balloon 120A in an eighth opening 114H via an eighth lumen 112H within the elongated member 110. In some embodiments, the eighth opening 114H is positioned at or adjacent to the distal end 110A of the elongated member 110, as shown in FIG. 1.

In some embodiments, a ninth lumen 112I of the plurality of lumens 112 extends at least partially through the elongated member 110. The ninth lumen 112I is configured to receive a stiffening member therein if it is desired that the elongated member 110 have increased rigidity.

In some embodiments, at least one inflation device 140 is connected to the catheter 100 at or adjacent to the proximal end 110B of the elongated member 110. The inflation device 140 is any device configured to deliver a flow of external fluid into the catheter 100, such as a fan, a compressed gas tank, a pump (e.g., an electric pump, a pneumatic pump, a hydraulic pump, a manual pump, etc.), oscillating membranes, a chemical reactor (e.g., a device that utilizes chemical reactions to generate flow), a user chargeable bladder or canister having a handheld bellows mechanism, chemical reactions, fans, pistons, or other electronic mechanisms, etc. A first inflation device 140A is in fluid communication with the second lumen 112B and is configured to deliver the first fluid into the second lumen 112B to inflate the first inflatable balloon 120A. A second inflation device 140B is in fluid communication with the third lumen 112C and is configured to deliver the second fluid into the third lumen 112C to inflate the second inflatable balloon 120B. In some embodiments, a third inflation device 140C is in fluid communication with the sixth lumen 112F and is configured to deliver the third fluid into the sixth lumen 112F to inflate the third inflatable balloon 120C. In some embodiments, a single inflation device 140 is in fluid communication with each of the second lumen 112B, the third lumen 112C, and the sixth lumen 112F and is configured to selectively deliver fluids to inflate each of the first inflatable balloon 120A, the second inflatable balloon 120B, and the third inflatable balloon 120C. In some embodiments, one or more of balloons 120A, 120B, 120C are inflated with the same type of fluid (e.g., air, water, etc.). In some embodiments, each balloon 120A, 120B, 120C is inflated with a different type of fluid (i.e., the first fluid, the second fluid, the third fluid).

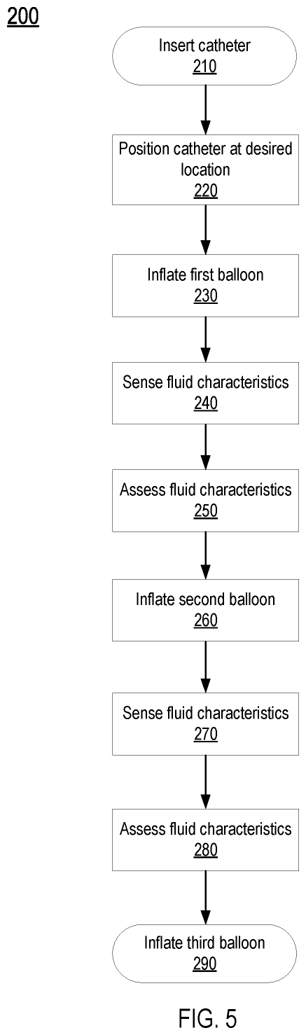

FIG. 5 illustrates a flowchart of a method 200 for blocking and/or removing an obstruction from a passageway according to some embodiments of the present technology. A 210, the method 200 includes inserting the catheter 100 described herein into a passageway. At 220, the method 200 includes positioning the catheter 100 in the passageway such that the distal end 110A of the elongated member 110 is at or adjacent to a desired location (e.g., at the desired blocking location or positioned such that the first inflatable balloon 120A is positioned past or distal from an obstruction in the passageway with the second inflatable balloon 120B positioned before or proximal from the obstruction). At 230, the method 200 includes inflating the first inflatable balloon 120A via the first inflation device 140A to block fluid flow in the passageway. At 240, the method 200 includes detecting, via the at least one first sensor 114A, the fluid characteristics in the passageway adjacent to the first inflatable balloon 120A. At 250, the method includes assessing fluid characteristics data received from the at least one first sensor 114A. In some embodiments, the fluid characteristics data is received at a display of a computing device that is connected to the catheter 100 or is in communication (e.g., via wired or wireless communication protocols) with the sensors 114 such that a user of the catheter 100 can view and assess the fluid characteristics data in real-time. At 260, the method includes inflating the second inflatable balloon 120B via the second inflation device 140B to further block fluid flow in the passageway if the fluid characteristics adjacent the first inflatable balloon 120A reaches a predetermined threshold. For example, if the fluid characteristics data indicates a pressure at or past the first inflatable balloon 120A that is higher than desired, the second inflatable balloon 120B is inflated as a precaution against the potential failure of the first inflatable balloon 120A.

In some embodiments, at 270, the method 200 includes detecting, via the at least one second sensor 114B, the fluid characteristics in the passageway adjacent to the second inflatable balloon 120B. At 280, the method 200 includes assessing fluid characteristics data received from the at least one second sensor 114B. At 290, the method 200 includes inflating the third inflatable balloon 120C via the third inflation device 140C to further block fluid flow in the passageway if the fluid characteristics adjacent the second inflatable balloon 120B reaches a predetermined threshold. In some embodiments, the method 200 includes detecting, via the at least one third sensor 114C, the fluid characteristics in the passageway adjacent to the third inflatable balloon 120C, and assessing fluid characteristics data received from the at least one third sensor 114C to monitor for the fluid characteristics adjacent to the third inflatable balloon 120C reaching a predetermined threshold.

In embodiments where the catheter 100 is used to remove an obstruction from the passageway, after inflating the first inflatable balloon 120A, the method includes inflating the second inflatable balloon 120B such that the obstruction is trapped in the first gap G1 between the first inflatable balloon 120A and the second inflatable balloon 120B, and removing the catheter 100 from the passageway with the obstruction trapped in the first gap G1.

As will be apparent to those skilled in the art, various modifications, adaptations, and variations of the foregoing specific disclosure can be made without departing from the scope of the technology claimed herein. The various features and elements of the technology described herein may be combined in a manner different than the specific examples described or claimed herein without departing from the scope of the technology. In other words, any element or feature may be combined with any other element or feature in different embodiments, unless there is an obvious or inherent incompatibility between the two, or it is specifically excluded.

References in the specification to “one embodiment,” “an embodiment,” etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described.

The singular forms “a,” “an,” and “the” include plural reference unless the context clearly dictates otherwise. Thus, for example, a reference to “a plant” includes a plurality of such plants. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation. The terms “preferably,” “preferred,” “prefer,” “optionally,” “may,” and similar terms are used to indicate that an item, condition, or step being referred to is an optional (not required) feature of the technology.

The term “and/or” means any one of the items, any combination of the items, or all of the items with which this term is associated. The phrase “one or more” is readily understood by one of skill in the art, particularly when read in context of its usage.

Each numerical or measured value in this specification is modified by the term “about.” The term “about” can refer to a variation of +5%, +10%, +20%, or +25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values and ranges proximate to the recited range that are equivalent in terms of the functionality of the composition, or the embodiment.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents of carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third, and upper third, etc.

As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the technology encompasses not only the entire group listed as a whole, but each member of the group individually and all possible subgroups of the main group. Additionally, for all purposes, the technology encompasses not only the main group, but also the main group absent one or more of the group members. The technology therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

What is claimed is:

1. A catheter comprising:
an elongated member with a plurality of lumens therein, the elongated member having a distal end and a proximal end defining a length of the elongated member, a first of the plurality of lumens extending to a first opening at the distal end of the elongated member;
a first inflatable balloon disposed about the elongated member adjacent the distal end, a second of the plurality of lumens extending to a second opening at an interior of the first inflatable balloon for inflating the first inflatable balloon;
a second inflatable balloon disposed about the elongated member at a position proximal from the first inflatable balloon forming a first gap between the first inflatable balloon and the second inflatable balloon, a third of the plurality of lumens extending to a third opening at an interior of the second inflatable balloon for inflating the second inflatable balloon;
a fourth of the plurality of lumens extending to a fourth opening in the first gap for positioning at least one first sensor; and
a fifth of the plurality of lumens extending to a fifth opening at a position proximal from the second inflatable balloon for positioning at least one second sensor.

2. The catheter of claim 1, further comprising at least one first sensor positioned in the fourth opening and at least one second sensor positioned in the fifth opening, wherein the at least one first sensor and at least one second sensor are configured to measure fluid characteristics in a passageway when the catheter is positioned in the passageway.

3. The catheter of claim 1, further comprising:
a third inflatable balloon disposed about the elongated member at a position proximal from the second inflatable balloon forming a second gap between the second inflatable balloon and the third inflatable balloon, a sixth of the plurality of lumens extending to a sixth opening at an interior of the third inflatable balloon for inflating the third inflatable balloon; and
a seventh of the plurality of lumens extending to a seventh opening at a position proximal from the third inflatable balloon for positioning at least one third sensor;
wherein the fifth opening for positioning the at least one second sensor via the fifth lumen is positioned in the second gap.

4. The catheter of claim 2, wherein each of the at least one first sensor and the at least one second sensor comprises pressure transducers, ultrasound transducers, sonic tips, flow sensors, or combinations thereof.

5. The catheter of claim 1, wherein the first lumen is configured for delivering at least one medication or medical tool to a position distal from the distal end of the elongated member.

6. The catheter of claim 1, further comprising an eighth of the plurality of lumens extending to an eighth opening at the distal end of the elongated member for positioning at least one fourth sensor.

7. The catheter of claim 1, further comprising a ninth of the plurality of lumens extending at least partially through the elongated member for receiving a stiffening member therein.

8. The catheter of claim 1, wherein at least two of the plurality of lumens are parallel to each other in a side-by-side arrangement.

9. The catheter of claim 1, wherein at least two of the plurality of lumens are in a coaxial arrangement.

10. A system for blocking a passageway, the system comprising:
a catheter configured for insertion into a passageway, the catheter comprising:
an elongated member with a plurality of lumens therein, the elongated member having a distal end and a proximal end defining a length of the elongated member, a first of the plurality of lumens extending to a first opening at the distal end of the elongated member;
a first inflatable balloon disposed about the elongated member adjacent the distal end, a second of the plurality of lumens extending to a second opening at an interior of the first inflatable balloon for inflating the first inflatable balloon;
a second inflatable balloon disposed about the elongated member at a position proximal from the first inflatable balloon forming a first gap between the first inflatable balloon and the second inflatable balloon, a third of the plurality of lumens extending to a third opening at an interior of the second inflatable balloon for inflating the second inflatable balloon;
a fourth of the plurality of lumens extending to a fourth opening in the first gap for positioning at least one first sensor; and
a fifth of the plurality of lumens extending to a fifth opening at a position proximal from the second inflatable balloon for positioning at least one second sensor;
a first inflation device in fluid communication with the second lumen, the first inflation device configured to deliver a first fluid into the second lumen to inflate the first inflatable balloon and block fluid flow in the passageway; and
a second inflation device in fluid communication with the third lumen, the second inflation device configured to deliver a second fluid into the second lumen to inflate the second inflatable balloon and block fluid flow in the passageway.

11. The system of claim 10, further comprising at least one first sensor positioned in the fourth opening and at least one second sensor positioned in the fifth opening, wherein the at least one first sensor and at least one second sensor are configured to measure fluid characteristics in a passageway when the catheter is positioned in the passageway.

12. The system of claim 10, wherein the catheter further comprises:
a third inflatable balloon disposed about the elongated member at a position proximal from the second inflatable balloon forming a second gap between the second inflatable balloon and the third inflatable balloon, a sixth of the plurality of lumens extending to a sixth opening at an interior of the third inflatable balloon for inflating the third inflatable balloon; and
a seventh of the plurality of lumens extending to a seventh opening at a position proximal from the third inflatable balloon for positioning at least one third sensor, the at least one third sensor is configured to measure fluid characteristics in the passageway;
wherein the fifth opening for positioning the at least one second sensor via the fifth lumen is positioned in the second gap.

13. The system of claim 12, further comprising a third inflation device in fluid communication with the sixth lumen, the third inflation device configured to deliver a third fluid into the sixth lumen to inflate the third inflatable balloon and block fluid flow in the passageway.

14. The system of claim 12, wherein each of the at least one first sensor, the at least one second sensor, and the at least one third sensor comprises pressure transducers, ultrasound transducers, sonic tips, flow sensors, or combinations thereof.

15. The system of claim 10, wherein the catheter further comprises an eighth of the plurality of lumens extending to an eighth opening at the distal end of the elongated member for positioning at least one fourth sensor.

16. The system of claim 10, wherein the catheter further comprises a ninth of the plurality of lumens extending at least partially through the elongated member for receiving a stiffening member therein.

17. A method for blocking a passageway, the method comprising:
inserting a catheter into a passageway, the catheter comprising:
an elongated member with a plurality of lumens therein, the elongated member having a distal end and a proximal end defining a length of the elongated member, a first of the plurality of lumens extending to a first opening at the distal end of the elongated member;
a first inflatable balloon disposed about the elongated member adjacent the distal end, a second of the plurality of lumens extending to a second opening at an interior of the first inflatable balloon for inflating the first inflatable balloon;
a second inflatable balloon disposed about the elongated member at a position proximal from the first inflatable balloon forming a first gap between the first inflatable balloon and the second inflatable balloon, a third of the plurality of lumens extending to a third opening at an interior of the second inflatable balloon for inflating the second inflatable balloon;
a fourth of the plurality of lumens extending to a fourth opening in the first gap for positioning at least one first sensor; and
a fifth of the plurality of lumens extending to a fifth opening at a position proximal from the second inflatable balloon for positioning at least one second sensor;
wherein each of the at least one first sensor and the at least one second sensor are configured to measure fluid characteristics in the passageway;
positioning the catheter in the passageway such that the distal end of the elongated member is adjacent a desired location; and
inflating, via a first inflation device in fluid communication with the first inflatable balloon, the first inflatable balloon with a first fluid to block fluid flow in the passageway.

18. The method of claim 17, further comprising:
detecting, via the at least one first sensor, fluid characteristics in the passageway adjacent the first inflatable balloon;
assessing fluid characteristics data received from the at least one first sensor; and
inflating, via a second inflation device in fluid communication with the second inflatable balloon, the second inflatable balloon with a second fluid to block fluid flow in the passageway if the fluid characteristics adjacent the first inflatable balloon reaches a predetermined threshold.

19. The method of claim 18, wherein the catheter further comprises:
a third inflatable balloon disposed about the elongated member at a position proximal from the second inflatable balloon forming a second gap between the second inflatable balloon and the third inflatable balloon, a sixth of the plurality of lumens extending to a sixth opening at an interior of the third inflatable balloon for inflating the third inflatable balloon; and
a seventh of the plurality of lumens extending to a seventh opening at a position proximal from the third inflatable balloon for positioning at least one third sensor, the at least one third sensor is configured to measure fluid characteristics in the passageway;
wherein the fifth opening for positioning the at least one second sensor via the fifth lumen is positioned in the second gap.

20. The method of claim 19, further comprising:
detecting, via the at least one second sensor, fluid characteristics in the passageway adjacent the second inflatable balloon;
assessing fluid characteristics data received from the at least one second sensor; and
inflating, via a third inflation device in fluid communication with the third inflatable balloon, the third inflatable balloon with a third fluid to block fluid flow in the passageway if the fluid characteristics adjacent the second inflatable balloon reaches a predetermined threshold.

21. The method of claim 20, further comprising:
detecting, via the at least one third sensor, fluid characteristics in the passageway adjacent the third inflatable balloon; and
assessing fluid characteristics data received from the at least one third sensor to monitor for the fluid characteristics adjacent the third inflatable balloon reaching a predetermined threshold.

22. The method of claim 17, wherein the catheter is positioned in the passageway such that the first inflatable balloon is positioned distal from an obstruction in the passageway and the second inflatable balloon is positioned proximal from the obstruction; the method further comprising:
inflating, via a second inflation device in fluid communication with the second inflatable balloon, the second inflatable balloon with a second fluid to trap the obstruction in the first gap between the first inflatable balloon and the second inflatable balloon; and
removing the catheter from the passageway with the obstruction trapped in the first gap.
